SEQUENCE LISTING (20 sequences)

SEQ ID NO: 1 - DNA, 20 bp, synthetic construct
  source 1..20: mol_type = other DNA
ccatgctcagacaggatgtg

SEQ ID NO: 2 - DNA, 20 bp, synthetic construct
  source 1..20: mol_type = other DNA
ttcctgtcccaagatttgct

SEQ ID NO: 3 - DNA, 20 bp, synthetic construct
  source 1..20: mol_type = other DNA
ccaattgtgttgaaggcacc

SEQ ID NO: 4 - DNA, 23 bp, synthetic construct
  source 1..23: mol_type = other DNA
ccgtgatgatgtggataaaatgt

SEQ ID NO: 5 - DNA, 20 bp, synthetic construct
  source 1..20: mol_type = other DNA
tgtgatgcagccatcgaact

SEQ ID NO: 6 - DNA, 20 bp, synthetic construct
  source 1..20: mol_type = other DNA
tgcatttggggaggtcagtc

SEQ ID NO: 7 - DNA, 21 bp, synthetic construct
  source 1..21: mol_type = other DNA
cgtctcgtaagagcgaacttg

SEQ ID NO: 8 - DNA, 22 bp, synthetic construct
  source 1..22: mol_type = other DNA
cgatgtatttctctccctggtc

SEQ ID NO: 9 - DNA, 22 bp, synthetic construct
  source 1..22: mol_type = other DNA
tgcagacatcgagtcctccaac

SEQ ID NO: 10 - DNA, 22 bp, synthetic construct
  source 1..22: mol_type = other DNA
gcttagcactggagactcgttg

SEQ ID NO: 11 - DNA, 21 bp, synthetic construct
  source 1..21: mol_type = other DNA
tcattacggtcacaatgacga

SEQ ID NO: 12 - DNA, 20 bp, synthetic construct
  source 1..20: mol_type = other DNA
gagtatctgctttccacggc

SEQ ID NO: 13 - DNA, 20 bp, synthetic construct
  source 1..20: mol_type = other DNA
aacaagtctttggccatcac

SEQ ID NO: 14 - DNA, 20 bp, synthetic construct
  source 1..20: mol_type = other DNA
tactcgttcagcacggtgac

SEQ ID NO: 15 - DNA, 20 bp, synthetic construct
  source 1..20: mol_type = other DNA
tcttgccaatttcagccttt

SEQ ID NO: 16 - DNA, 20 bp, synthetic construct
  source 1..20: mol_type = other DNA
gcacaacgatcacaacatcc

SEQ ID NO: 17 - DNA, 20 bp, synthetic construct
  source 1..20: mol_type = other DNA
ccattctctgccaggttagc

SEQ ID NO: 18 - DNA, 20 bp, synthetic construct
  source 1..20: mol_type = other DNA
acccctaatcagcacagtgg

SEQ ID NO: 19 - DNA, 20 bp, synthetic construct
  source 1..20: mol_type = other DNA
tcacgaagctcatcatccag

SEQ ID NO: 20 - DNA, 20 bp, synthetic construct
  source 1..20: mol_type = other DNA
ttcctcagggactcatcacc

(19) United States
(12) Patent Application Publication
(10) Pub. No.: US 2025/0051729 A1
(43) Pub. Date: Feb. 13, 2025

(54) METHOD FOR PREPARING HIGHLY DIFFERENTIATED KIDNEY ORGANOIDS

(71) Applicant: RGENORGANO BIOTECHNOLOGY CO., LTD., Seoul (KR)

(72) Inventors: Yong Kyun KIM, Gyeonggi-do (KR); Sun Ah NAM, Gyeonggi-do (KR); Jin Won KIM, Seoul (KR)

(21) Appl. No.: 18/719,518

(22) PCT Filed: Oct. 24, 2022
(86) PCT No.: PCT/KR2022/016267

§ 371 Date: Jun. 13, 2024

(30) Foreign Application Priority Data
Dec. 13, 2021 (KR) 10-2021-0178114

(51) Int. Cl. C12N 5/071

(52) U.S. Cl. CPC C12N 5/0686; C12N 2501/15; C12N 2501/165; C12N 2501/727; C12N 2506/03; C12N 2513/00; C12N 2533/54; C12N 2533/90

(57) ABSTRACT

The present invention relates to: a method form preparing highly differentiated kidney organoids; and the like. The present applications have developed two protocols for preparing kidney organoids by arranging stem cells between kidney decellularized extracellular matrix and Matrigel and uniquely treating same with a composition for differentiating kidney organoids, and has identified that these protocols promote the formation of a vascular network and promote glomerular vascularization in kidney organoids, and also have the excellent effect of maturating kidney organoids, and thus it is expected that kidney organoids according to the present invention can be effectively usable in disease modeling, drug screening, regenerative medicine, and the like.

### TECHNICAL FIELD

The present invention relates to a method of preparing highly differentiated kidney organoids.

This application claims priority to and the benefit of Korean Patent Application No. 10-2021-0178114, filed on Dec. 13, 2021, the disclosure of which is incorporated herein by reference in its entirety.

### BACKGROUND ART

Recent advances in stem cell biology have established several protocols for producing kidney organoids from human pluripotent stem cells (hPSCs). These hPSC-derived kidney organoids include segmented structures with podocytes, proximal tubules, and distal tubules in a nephron-like structure, similar to a real human kidney. Recent studies have revealed that hPSC-kidney organoids can recapitulate the development of human kidneys and mimic various kidney diseases. In the future, hPSC-kidney organoids are a promising cell source for kidney tissue for kidney tissue regeneration and repair, and are expected to be applied as a therapeutic tool in various kidney disease models. However, despite recent technological advances, the clinical application of kidney organoids differentiated from hPSCs has challenges to overcome, including safety, immaturity, and limited vascularization of kidney organoids. That is, existing protocols cannot produce kidney organoids that fully recapitulate complicated structure and functions of the kidney, limiting their application in both kidney disease modeling and regenerative medicine. Therefore, for clinical application of kidney organoids, it is necessary to develop a differentiation protocol for kidney organoids that are highly differentiated and have a vascular network.

Kidney decellularized extracellular matrix (kidney dECM) is expected to improve the survival, proliferation, and differentiation of cultured cells and provide a specialized environment for regeneration of artificial organs such as kidney organoids by providing a three-dimensional microenvironment similar to that in intact tissue in which various ECM components including collagen, fibronectin, and laminin are preserved in decellularized tissue.

Through the development of unique and appropriate treating methods for kidney dECM, a vascular growth factor, Compound 1, the present inventors completed the invention of a 3D culture protocol for kidney organoids with a much more developed vascular network compared to existing hPSC-kidney organoids, and greatly enhanced the maturation of podocytes and tubular cells. Accordingly, the kidney organoids prepared using the protocol developed by the present inventors are expected to be used in regenerative medicine, kidney drug toxicity testing, and development of new drugs for intractable kidney disease.

### DISCLOSURE

### Technical Problem

Therefore, as a result of diligent efforts to produce highly differentiated kidney organoids, the present inventors developed a unique treating method for kidney decellularized extracellular matrix (dECM), Matrigel, and a composition for kidney organoid differentiation, thereby producing kidney organoids, and completed the present invention.

The present invention is directed to providing a method of preparing highly differentiated kidney organoids, which includes coating a culture container with kidney dECM: placing stem cells in the coated culture container: adding Matrigel; and differentiating the stem cells into kidney organoids using a composition for organoid differentiation.

The present invention is also directed to providing a kit for preparing highly differentiated kidney organoids, which includes a first kit, a second kit, and instructions, wherein the first kit includes kidney dECM, stem cells, and Matrigel, the second kit includes a composition for organoid differentiation, and the instructions includes a step of sequentially treating the first and second kits.

The present invention is also directed to providing highly differentiated kidney organoids, which are prepared by the above-described method and satisfy the following characteristics:
(a) promotion of the formation of a vascular network of kidney organoids;
(b) promotion of glomerular angiogenesis;
(c) promotion of kidney organoid morphogenesis; and
(d) enhancement of kidney organoid maturity.

The present invention is also directed to providing a method of preparing a biomimetic organ model capable of simulating the vasculopathy in Fabry disease, which includes coating a culture container with kidney dECM; placing stem cells in which the alpha-galactosidase (GLA) gene has been knocked out in the coated culture container: adding Matrigel; and differentiating the stem cells into kidney organoids using a composition for organoid differentiation.

However, technical problems to be solved in the present invention are not limited to the above-described problems, and other problems which are not described herein will be fully understood by those of ordinary skill in the art from the following descriptions.

### Technical Solution

To achieve the purposes of the present invention, the present invention provides a method of preparing highly differentiated kidney organoids, which includes coating a culture container with kidney dECM; placing stem cells in the coated culture container; adding Matrigel; and differentiating the stem cells into kidney organoids using a composition for organoid differentiation.

In addition, the present invention provides a kit for preparing highly differentiated kidney organoids, which includes a first kit, a second kit, and instructions, wherein the first kit includes kidney dECM, stem cells, and Matrigel, the second kit includes a composition for organoid differentiation, and the instructions includes a step of sequentially treating the first and second kits.

In addition, the present invention provides highly differentiated kidney organoids which are prepared by the above-described method and satisfy the following characteristics:
(a) promotion of the formation of a vascular network of kidney organoids;
(b) promotion of glomerular angiogenesis;
(c) promotion of kidney organoid morphogenesis; and
(d) enhancement of kidney organoid maturity.

In addition, the present invention provides a method of preparing a biomimetic organ model capable of simulating the vasculopathy in Fabry disease, which includes coating a culture container with kidney dECM: placing stem cells in which the GLA gene is knocked out in the coated culture container; adding Matrigel; and differentiating the stem cells into kidney organoids using a composition for organoid differentiation.

In one embodiment of the present invention, the differentiating of the stem cells into kidney organoids may include adding a glycogen synthase kinase 3 (GSK3) inhibitor, and providing a medium for differentiation, but the present invention is not limited thereto.

In another embodiment of the present invention, the differentiating of the stem cells into kidney organoids may include adding a vascular endothelial growth factor (VEGF); and adding a TGF-β inhibitor, but the present invention is not limited thereto.

In still another embodiment of the present invention, the stem cells may be placed between the kidney dECM and the Matrigel, but the present invention is not limited thereto.

In yet another embodiment of the present invention, the coating step may be coating each well with the kidney dECM, but the present invention is not limited thereto.

In yet another embodiment of the present invention, the method may satisfy the following characteristics, but the present invention is not limited thereto:
(a) promotion of the formation of a vascular network of kidney organoids;
(b) promotion of glomerular angiogenesis;
(c) promotion of kidney organoid morphogenesis; and
(d) enhancement of kidney organoid maturity.

In yet another embodiment of the present invention, the method may increase the expression of one or more genes selected from the group consisting of platelet endothelial cell adhesion molecule 1 (PECAM1), a melanoma cell adhesion molecule (MCAM), polycystin 1 (PKD1), polycystin 2 (PKD2), polycystic kidney and hepatic disease 1 (PKHD1), solute carrier family 34 member 1 (SLC34A1), ATPase Na+/K+ transporting subunit alpha 1 (ATP1A1), ATP binding cassette subfamily B member 1 (ABCB1), LDL receptor related protein 2 (LRP2), and GATA binding protein 3 (GATA3), but the present invention is not limited thereto.

In yet another embodiment of the present invention, the stem cells may be pluripotent stem cells, but the present invention is not limited thereto.

In yet another embodiment of the present invention, the composition for organoid differentiation may include two or more selected from the group consisting of a glycogen synthase kinase 3 (GSK3) inhibitor, a vascular endothelial growth factor (VEGF), a TGF-β inhibitor, and a medium for differentiation, but the present invention is not limited thereto.

### Advantageous Effects

The present inventors invented two protocols for preparing kidney organoids by placing stem cells between kidney dECM and Matrigel and uniquely treating a composition for kidney organoids, and since it was confirmed that these protocols not only promote the formation of a vascular network of kidney organoids, promote glomerular angiogenesis, but also exhibit the excellent effect of maturing kidney organoids, it is expected that the kidney organoids prepared according to the present invention can be effectively used in disease modeling, drug screening, and regenerative medicine.

### DESCRIPTION OF DRAWINGS

FIG. 1A shows protocols for kidney organoid differentiation based on a kidney decellularized extracellular matrix (dECM) hydrogel.

FIG. 1B shows a set of bright field images of the morphologies of kidney organoids cultured using a Matrigel-based protocol, Protocol A, and Protocol B (scale bar=200 μm).

FIG. 1C shows the diameters of kidney organoids cultured using Matrigel-based protocol, Protocol A, and Protocol B.

FIGS. 1D and 1E show a set of confocal images of PECAM1, and a graph of PECAM1 positive areas (scale bar=50 μm).

FIGS. 1F and 1G show a set of confocal images and a graph of vessel diameters of PECAM1 and kidney organoids (scale bar=25 μm).

FIG. 1H shows the gene expression of MCAM, and PECAM1 using a Matrigel-based protocol, Protocol A, and Protocol B through RT-qPCR (values are mean±SEM: *, p<0.05: **, p<0.01: ***, p<0.001: ****, and p<0.0001).

FIG. 2A shows a set of images of immunofluorescent staining of NPHS1 (podocytes), PECAM1 (vascular network), and laminin (basement membrane) in kidney organoids cultured using a Matrigel-based protocol, Protocol A, and Protocol B (white arrows: invasion of PECAM1+ endothelial cells into podocyte clusters: scale bar=20 μm).

FIGS. 2B and 2C are a set of images of immunofluorescent staining of WT1 (podocytes), PECAM1 (vascular network), and collagen IV (basement membrane) in kidney organoids cultured using Protocol B (white arrows: invasion of PECAM1+ endothelial cells into WT1+ podocyte clusters: the scale bar of FIG. 2B=20 μm; the scale bar of FIG. 2C=100 μm).

FIG. 2D shows the results of correlative light-and-electron microscopy (CLEM) performed by overlapping confocal microscope images of stained PECAM1, WT1, and collagen IV and electron microscope (EM) images with the same structure of kidney organoids (scale bar =20 μm).

FIGS. 3A to 3C show images of immunofluorescent staining of LTL, collagen IV, TUBA4A, and acetylated tubulin in kidney organoids cultured using a Matrigel-based protocol, and Protocol A (the scale bars of FIGS. 3A and 3C=20 μm; the scale bar of FIG. 3B=50 μm).

FIG. 3D shows qRT-PCR analysis results for PKD1, PKD2, and PKHD1 (ciliary genes); SLC34A1 and ATP1A1 (tubular epithelial transport genes): ABCB1 and LRP2 (drug transport gene) (values are mean±SEM: *, p<0.05; **, p<0.01: ***, and p<0.001).

FIG. 3E shows a set of images of immunofluorescent staining of CK8, GATA3, and LTL in kidney organoids cultured using Protocol A (scale bar=50 μm).

FIG. 3F shows the results of confirming the expression level of GATA3 through uniform manifold approximation and projection (UMAP).

FIG. 4A shows the results of confirming GLA expression in GLA mutant 1 and GLA mutant 2 through Western blotting.

FIG. 4B shows images of immunofluorescent staining of NPHS1 and LTL in wild-type and GLA mutant kidney organoids (scale bar=50 μm).

FIG. 4C shows the results of confirming the accumulation of lipid droplets, glycoproteins, and zebra bodies using TEM images (scale bar=1 μm).

FIG. 4D shows a set of images of immunofluorescent staining of Gb3 in a wild type, GLA mutant kidney organoids, and human recombinant agalsidase-α-treated GLA mutant kidney organoids (scale bar=50 μm).

FIG. 4E is a graph showing Gb3 positive areas in a wild type, GLA mutant kidney organoids, and human recombinant agalsidase-α-treated GLA mutant kidney organoids.

FIG. 4F is a set of images of immunofluorescent staining of NPHS1, PECAM1, and LTL in a wild type, GLA mutant kidney organoids, and human recombinant agalsidase-α-treated GLA mutant kidney organoids (white arrow: invasion of PECAM1+ endothelial cells into the NPHS1+ podocyte structure: scale bar=50 μm).

### [Best Modes]

In one embodiment of the present invention, it was confirmed that, when kidney organoids are produced using Protocols A and B, the size of kidney organoids increases, the formation of a vascular network is promoted and the area and diameter of PECAM1 positive vasculature increase, and the gene expression of the vascular marker PECAM1 and its precursor MCAM increases (refer to Example 3).

In one embodiment of the present invention, it was confirmed that, when kidney organoids are produced using Protocols A and B, a glomerular angiogenesis effect is identified by confirming that a small branch of a PECAM1⁺ vessel-like structure invades an NPHS1⁺ or WT1⁺ glomerular structure (refer to Example 4).

In one embodiment of the present invention, it was confirmed that, when kidney organoids are produced using Protocol A, the expression of ciliary genes (PKD1, PKD2, PKHD1), tubular epithelial transport genes (SLC34A1, and ATPIA1), and drug transport genes (ABCB1, and LRP2) is upregulated, and the anterior intermediate mesoderm (AIM) marker GATA3 is highly expressed. Through this, the effect of maturing kidney organoids was confirmed (refer to Example 4).

In one embodiment of the present invention, Gb3 accumulation in GLA mutant kidney organoids prepared using Protocol B was confirmed, and the formation of concentric bodies (zebra bodies) in the cytoplasm of podocytes and tubules was observed. In addition, ERT using recombinant human agalsidase-α restored a vascular network by glomerular angiogenesis and the formation of large lumen vessels in GLA mutant kidney organoids, and it was confirmed that Protocol B is useful for modeling Fabry disease and developing treatment options (refer to Example 5).

Accordingly, the present inventors confirmed that the preparation method of the present invention is able to induce the vascularization and maturation of kidney organoids and able to be effectively used in disease modeling, drug screening, and regenerative medicine.

Hereinafter, the present invention will be described in detail.

The present invention provides a method of preparing highly differentiated kidney organoids, which includes: coating a culture container with kidney dECM: placing stem cells in the coated culture container; adding Matrigel; and differentiating the stem cells into kidney organoids using a composition for organoid differentiation.

In the present invention, the differentiating of the stem cells into kidney organoids may include adding a glycogen synthase kinase 3 (GSK3) inhibitor, and providing a medium for differentiation, but the present invention is not limited thereto.

In the coating step in the present invention, each well of the well-plate may be coated with kidney dECM, but the present invention is not limited thereto.

In the present invention, the concentration of the kidney dECM may be 0.01 to 50% (w/v), the concentration of the Matrigel may be 0.1 to 10%, and the concentration of the GSK3 inhibitor may be 1 to 50 μM, but the present invention is not limited thereto.

In the present invention, during the adding of Matrigel, the formation of dense ball-shaped colonies with developed internal cavities may be observed, but the present invention is not limited thereto. The medium for differentiation may be mTesr, RPMI, or B27-supplemented RPMI medium, and may be replaced every 1 to 7 days, but the present invention is not limited thereto.

In the present invention, the glycogen synthase kinase 3 (GSK3) inhibitor may be CHIR 99021, CHIR 98014, 3F8, A1070722, Alsterpaullone, or AR-A014418, and according to one embodiment, may be CHIR99021, but the present invention is not limited thereto.

In the present invention, according to the method, the kidney organoids may be prepared for 10 to 50 days, 10 to 40 days, 10 to 30 days, 10 to 20 days, 11 to 50 days, 11 to 40 days, 11 to 30 days, 11 to 20 days, 12 to 50 days, 12 to 40 days, 12 to 30 days, 12 to 20 days, 13 to 50 days, 13 to 40 days, 13 to 30 days, 13 to 20 days, 14 to 50 days, 14 to 40 days, 14 to 30 days, or 14 to 20 days, but the present invention is not limited thereto.

In the present invention, the differentiating of the stem cells into kidney organoids may include adding a vascular endothelial growth factor (VEGF); and adding a TGF-β inhibitor, but the present invention is not limited thereto.

In the present invention, the VEGF may be treated in divided portions 1 to several times, 1 to 15 times, 1 to 12 times, 1 to 10 times, 1 to 7 times, 1 to 5 times, 2 to 15 times, 2 to 12 times, 2 to 10 times, 2 to 7 times, 2 to 5 times, 3 to 15 times, 3 to 12 times, 3 to 10 times, 3 to 7 times, or 3 to 5 times during the period of organoid differentiation. According to one embodiment, the VEGF treatment may be performed four times, and particularly, the VEGF may be treated at 1 to 100 ng/ml on days 6, 9, 12, and 14 of organoid differentiation, but the present invention is not limited thereto.

In the present invention, the TGF-β inhibitor may be treated in divided portions 1 to several times, 1 to 15 times, 1 to 10 times, 1 to 7 times, 1 to 5 times, or 1 to 3 times during the period of organoid differentiation. According to one embodiment, the TGF-β inhibitor may be treated once, and particularly, treated at 1 to 50 μM on day 12 of organoid differentiation, but the present invention is not limited thereto.

The “decellularization” used herein refers to the removal of cell components except for ECM from cells or tissue, e.g., the nucleus, the cell membrane, and the nucleic acid. In addition, “decellularized extracellular matrix (ECM)” refers to the ECM remaining after the cell components such as the nucleus, the cell membrane, and the nucleic acid are removed from tissue or cells.

The “organoid” used herein refers to an organ-specific cell aggregate made by aggregating and recombining cells isolated from stem cells or organ progenitor cells through a 3D culture method, and an organ-specific cell-type organoid that self-organizes (or self-patterns) through cell sorting and spatially-limited lineage commitment in a manner similar to the in vivo state. Accordingly, organoids represent the native physiology of cell and have an anatomical structure that mimics the native state of a cell mixture (including not only limited cell types but also residual stem cells and proximal physiological niches). Stem cells may be isolated from tissue or organoid fragments. Cells from which organoids are produced differentiate to form organ-like tissue that exhibits multiple cell types that self-organize to form a structure very similar to an organ in vivo. Therefore, an organoid is an excellent model for studying human organs, and human organ development in a system very similar to in vivo development.

In the present invention, the high differentiation means promotion of the vascularization of kidney organoids, and maturation of kidney organoids, such as the maturation of the tubular compartment.

In the present invention, the kidney dECM may be prepared from kidney tissue of an animal excluding a human. In one embodiment of the present invention, kidney tissue obtained from a pig was used, but the present invention is not limited thereto.

The kidney dECM may be prepared by a method including the following steps, but the present invention is not limited thereto:
(a) sectioning kidney tissue of an animal excluding a human:
(b) treating Triton X-100 dissolved in NaCl:
(c) treating DNase:
(d) performing sterilization; and
(e) performing lyophilization.

In the present invention, the sections in (a) may have a thickness of 0.01 to 1 mm, but the present invention is not limited thereto.

In the present invention, the stem cells may be placed between the kidney dECM, and the Matrigel, but the present invention is not limited thereto. In addition, the stem cells may be pluripotent stem cells, but the present invention is not limited thereto.

The “stem cell” used herein is a cell that can differentiate into various cells, which constitute biological tissue, refers to undifferentiated cells capable of being unlimitedly regenerated to form specialized cells of tissue and organs. A stem cell is a pluripotent or multipotent cell capable of development, and may divide into two daughter stem cells, or one daughter stem cell and one transit cell, and then proliferate into mature and complete forms of cells in tissue.

The “pluripotent stem cell” used herein refers to a stem cell in a more advanced state of development than a fertilized egg and can differentiate into cells that constitute the endoderm, the mesoderm, and the ectoderm. For example, the pluripotent stem cells may be embryonic stem cells, embryonic germ cells, embryonic carcinoma cells, or induced pluripotent stem cells, and according to one embodiment, may be induced pluripotent stem cells (iPSCs), but the present invention is not limited thereto.

The “vascular endothelial growth factor (VEGF)” used herein is a glycoprotein that specifically acts on vascular endothelial cells to promote cell proliferation or angiogenesis. It may promote the formation of a vascular network.

The “transforming growth factor β (TGF-β) inhibitor” used herein is a multifunctional cytokine that regulates the growth, migration, differentiation, and death of epithelial cells and hematopoietic cells. It may enhance podocyte differentiation. In one embodiment, a compound represented in Chemical Formula 1 below was used, but the present invention is not limited thereto.

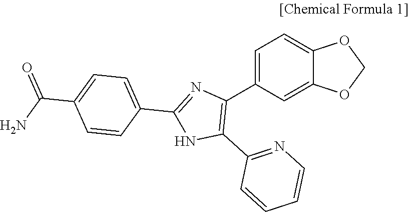 [SMILES: NC(=O)c1ccc(-c2nc(-c3ccc4c(c3)OCO4)c(-c3ccccn3)[nH]2)cc1]

The compound represented by Chemical Formula 1 may have the IUPAC name of 4-[4-(1,3-benzodioxol-5-yl)-5-pyridin-2-yl-1H-imidazol-2-yl]benzamide, its chemical formula may be C₂₂H₁₆N₄O₃, and its molecular weight may be 384.4, but the present invention is not limited thereto. In addition, the compound may be referred to as Compound 1.

In the present invention, the method may satisfy the following characteristics, but the present invention is not limited thereto:
(a) promotion of the formation of a vascular network of kidney organoids;
(b) promotion of glomerular angiogenesis;
(c) promotion of kidney organoid morphogenesis; and
(d) enhancement of kidney organoid maturity.

In the present invention, the method may increase the expression of one or more genes selected from the group consisting of a vascular marker such as platelet endothelial cell adhesion molecule 1 (PECAM1); the precursor of a vascular marker such as a melanoma cell adhesion molecule (MCAM); ciliary genes such as polycystin 1 (PKD1), polycystin 2 (PKD2), and polycystic kidney and hepatic disease 1 (PKHD1); tubular epithelial transport genes such as solute carrier family 34 member 1 (SLC34A1) and ATPase Na+/K+ transporting subunit alpha 1 (ATP1A1); drug transport genes such as ATP binding cassette subfamily B member 1 (ABCB1) and LDL receptor related protein 2 (LRP2); and an anterior intermediate mesoderm (AIM) marker such as GATA binding protein 3 (GATA3), but the present invention is not limited thereto.

The present invention provides a kit for preparing highly differentiated kidney organoids, which includes a first kit, a second kit, and instructions, wherein
the first kit includes kidney dECM, stem cells, and Matrigel, the second kit includes a composition for organoid differentiation, and the instructions includes a step of sequentially treating the first and second kits.

In the present invention, the instructions may include coating a culture container with kidney dECM: placing stem cells in the coated culture container: adding Matrigel; and differentiating the stem cells into kidney organoids using a composition for organoid differentiation, but the present invention is not limited thereto.

In the present invention, the composition for organoid differentiation may include two or more selected from the group consisting of a glycogen synthase kinase 3 (GSK3) inhibitor, a vascular endothelial growth factor (VEGF), a TGF-β inhibitor, and a medium for differentiation, but the present invention is not limited thereto.

In the present invention, organoid preparation includes all actions that can generate or maintain organoids. For example, cells or cells isolated from specific tissue may differentiate into tissue or organ cells with a specific function, or organoids may survive, grow, or proliferate.

In addition, the present invention provides highly differentiated kidney organoids, which are prepared by the above-described method and satisfy the following characteristics:
(a) promotion of the formation of a vascular network of kidney organoids;
(b) promotion of glomerular angiogenesis;
(c) promotion of kidney organoid morphogenesis; and
(d) enhancement of kidney organoid maturity.

In the present invention, the kidney organoids may be used in disease modeling, biomimetic organ models, or drug screening, but the present invention is not limited thereto. The drug screening may screen a drug tailored to the characteristics of a patient's disease, but the present invention is not limited thereto.

The kidney organoids may be used to treat a subject in vivo. The present invention includes a method of treating a kidney disease in a subject. As disclosed in the present invention, in one aspect, the method includes improving, treating, or alleviating symptoms of a kidney disease in a subject in need thereof. The method also includes administering kidney organoids in an amount effective for treating a kidney disease in the subject.

The treatment method for the subject may further include administering kidney organoids, or transplanting kidney organoids, and here, as a result of administration or transplantation, the symptoms of a kidney disease in the subject are improved, treated, or alleviated. The improvement, treatment, or alleviation may be any change in the kidney disease or symptoms of the kidney disease, which can be detected using natural senses or artificial devices.

The “subject” or “patient” as used in the present invention may be a human or non-human mammal. The non-human mammal includes, for example, livestock and pets, such as sheep, cattle, pigs, dogs, cats, and murine mammals. Preferably, the subject is a human.

The kidney organoids of the present invention may be included in the pharmaceutical composition in an amount of 1 μg to 30 g w/v %, but the present invention is not limited thereto.

The “individual” used herein is not limited to vertebrates, but may specifically be applied to humans, mice, rats, guinea pigs, rabbits, monkeys, pigs, horses, cows, sheep, antelopes, dogs, and cats, and preferably, humans.

The “administration” used herein refers to introducing the pharmaceutical composition of the present invention to a patient in any appropriate way, and the composition of the present invention may be administered via various oral or parenteral routes as long as it can reach target tissue.

The “prevention” used herein refers to all actions that delay kidney diseases by administration of the composition according to the present invention, the “treatment” used herein refers to all actions involved in improving or beneficially changing the symptoms of kidney diseases by administration of the composition according to the present invention, the “prevention” used herein refers to all actions that reduce the degree of parameters associated with kidney diseases, e.g., symptoms, by administration of the composition according to the present invention.

The content of the kidney organoids in the composition of the present invention may be appropriately adjusted depending on the symptoms of a disease, the degree of progression of the symptoms, and a patient's condition, and for example, may be 0.0001 to 99.9 wt %, or 0.001 to 50 wt % based on the total weight of the composition, but the present invention is not limited thereto. The content ratio is the value based on a dry amount from which a solvent is removed.

The pharmaceutical composition according to the present invention may further include appropriate carriers, excipients and diluents, which are generally used in the preparation of pharmaceutical compositions. The excipient may be, for example, one or more selected from the group consisting of a diluent, a binder, a disintegrant, a lubricant, an adsorbent, a humectant, a film-coating material, and a controlled-release additive.

The pharmaceutical composition according to the present invention may be formulated and used in the form of a powder, a granule, a sustained-release granule, an enteric granule, a liquid, an ophthalmic solution, an elixir, an emulsion, a suspension, a spirit, a troche, aromatic water, a lemonade, a tablet, a sustained-release tablet, an enteric tablet, a sublingual tablet, a hard capsule, a soft capsule, a sustained-release capsule, an enteric capsule, a pill, a tincture, a soft extract, a dry extract, a fluid extract, an injection, a capsule, a perfusate, a plaster, a lotion, a paste, a spray, an inhalant, a patch, a sterile injection, or an external preparation such as an aerosol according to a conventional method, and the external preparation may be formulated as a cream, a gel, a patch, a spray, an ointment, a plaster, a lotion, a liniment, a paste or a cataplasma.

Carriers, excipients, and diluents, which can be included in the pharmaceutical composition according to the present invention, may include lactose, dextrose, sucrose, an oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxy benzoate, propyl hydroxy benzoate, talc, magnesium stearate, and mineral oil.

During preparation, commonly used diluents or excipients, such as a filler, an extender, a binder, a wetting agent, a disintegrant, and a surfactant, may be used.

Additives for tablets, powders, granules, capsules, pills, and troches may include excipients such as corn starch, potato starch, wheat starch, lactose, sucrose, glucose, fructose, di-mannitol, precipitated calcium carbonate, synthetic aluminum silicate, calcium monohydrogen phosphate, calcium sulfate, sodium chloride, sodium bicarbonate, purified lanolin, microcrystalline cellulose, dextrin, sodium alginate, methylcellulose, carboxymethylcellulose, sodium carboxymethylcellulose, kaolin, urea, colloidal silica gel, hydroxypropyl starch, hydroxypropyl methyl cellulose (HPMC) 1928, HPMC 2208, HPMC 2906, HPMC 2910, propylene glycol, casein, calcium lactate, and Primogel: binders such as gelatin, gum arabic, ethanol, agar powder, cellulose acetate phthalate, carboxymethyl cellulose, carboxymethyl cellulose calcium, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch powder, hydroxypropylcellulose, hydroxypropylmethylcellulose, polyvinyl alcohol, and polyvinylpyrrolidone: disintegrants such as hydroxypropylmethylcellulose, corn starch, agar powder, methylcellulose, bentonite, hydroxypropyl starch, sodium carboxymethylcellulose, calcium citrate, sodium lauryl sulfate, silicic anhydride, 1-hydroxypropyl cellulose, dextran, an ion exchange resin, polyvinyl acetate, formaldehyde-treated casein and gelatin, alginic acid, amylose, guar gum, sodium bicarbonate, polyvinylpyrrolidone, calcium phosphate, gelled starch, gum arabic, amylopectin, pectin, sodium polyphosphate, ethyl cellulose, sucrose, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, petrolatum, sodium stearate, cacao butter, sodium salicylate, magnesium salicylate, polyethylene glycol (PEG) 4000, PEG 6000, liquid paraffin, hydrogenated soy bean oil (Lubri wax), aluminum stearate, zinc stearate, sodium lauryl sulfate, magnesium oxide, Macrogol, synthetic aluminum silicate, silicic anhydride, a higher fatty acid, a higher alcohol, silicone oil, paraffin oil, polyethylene glycol fatty acid ether, starch, sodium chloride, sodium acetate, sodium oleate, dileucine, and light anhydrous silicic acid.

Additives for a liquid formulation according to the present invention may include water, diluted hydrochloric acid, diluted sulfuric acid, sodium citrate, monostearate sucrose, polyoxyethylene sorbitol fatty acid esters (Tween esters), polyoxyethylene monoalkylethers, lanolin ethers, lanolin esters, acetic acid, hydrochloric acid, acetic acid, hydrochloric acid, aqueous ammonia, ammonium carbonate, potassium hydroxide, sodium hydroxide, prolamine, polyvinylpyrrolidone, ethyl cellulose, and sodium carboxymethylcellulose.

A syrup according to the present invention may include a solution of white sugar, a different type of sugar, or a sweetener, and if necessary, a fragrance, a colorant, a preservative, a stabilizer, a suspending agent, an emulsifier, and a thickener.

An emulsion according to the present invention may include distilled water, and if necessary, may include an emulsifier, a preservative, a stabilizer, and a fragrance.

A suspension according to the present invention may include a suspending agent such as acacia, tragacanth, methylcellulose, carboxymethylcellulose, sodium carboxymethylcellulose, microcrystalline cellulose, sodium alginate, hydroxypropylmethyl cellulose (HPMC), HPMC 1828, HPMC 2906, or HPMC 2910, and if necessary, a surfactant, a preservative, a stabilizer, a colorant, or a fragrance.

An injection according to the present invention may include a solvent such as injectable sterile water, 0.9% sodium chloride for injection, Ringer's solution, dextrose for injection, dextrose+ sodium chloride for injection, PEG, lactated Ringer's solution, ethanol, propylene glycol, non-volatile oil-sesame oil, cottonseed oil, peanut oil, soybean oil, corn oil, ethyl oleate, isopropyl myristic acid, or benzene benzoate; a solubilizing agent such as sodium benzoate, sodium salicylate, sodium acetate, urea, urethane, monoethylacetamine, butazolidine, propylene glycol, Tween, nicotinamide, hexamine or dimethylacetamide; a buffer such as a weak acid and a salt thereof (acetic acid and sodium acetate), a weak base and a salt thereof (ammonia and ammonium acetate), an organic compound, a protein, albumin, peptone, or gums; an isotonic agent such as sodium chloride: a stabilizer such as sodium bisulfite (NaHSO₃), carbon dioxide gas, sodium metabisulfite (Na₂S₂O₅), sodium sulfite (Na₂SO₃), nitrogen gas (N₂), or ethylenediaminetetracetic acid: an antioxidant such as sodium bisulfide 0.1%, sodium formaldehyde sulfoxylate, thiourea, disodium ethylenediaminetetraacetate, or acetone sodium bisulfite; an analgesic such as benzyl alcohol, chlorobutanol, procaine hydrochloride, glucose, or calcium gluconate; or a suspending agent such as sodium CMC, sodium alginate, Tween 80, or aluminum monostearate.

A suppository according to the present invention may include a base such as cacao butter, lanolin, Witepsol, polyethylene glycol, glycerogelatin, methyl cellulose, carboxymethylcellulose, a mixture of stearate and oleate, Subanal, cottonseed oil, peanut oil, palm oil, cacao butter+ cholesterol, lecithin, Lanette wax, glycerol monostearate, Tween or Span, Imhausen, monolene (propylene glycol monostearate), glycerin, Adeps solidus, Buytyrum Tego-G, Cebes Pharma 16, hexalide base 95, Cotomar, Hydrokote SP, S-70-XXA, S-70-XX75 (S-70-XX95), Hydrokote 25, Hydrokote 711, Idropostal, Massa estrarium, (A, AS, B, C, D, E, I, T), Mass-MF, Masupol, Masupol-15, neosuppostal-N, paramount-B, supposiro (OSI, OSIX, A, B, C, D, H, L), suppository base IV types (AB, B, A, BC, BBG, E, BGF, C, D, 299), Suppostal (N, Es), Wecoby (W, R, S, M,Fs), or a Tegester triglyceride base (TG-95, MA, 57).

Solid preparations for oral administration include tablets, pills, powders, granules, and capsules, and are formulated by mixing at least one excipient, such as starch, calcium carbonate, sucrose, lactose, or gelatin with an extract. Aside from simple excipients, lubricants such as magnesium stearate, and talc are also used.

Liquid preparations for oral administration include suspensions, liquids for internal use, emulsions, and syrups, and may further include various types of excipients, for example, a wetting agent, a sweetener, a fragrance and a preservative, other than a commonly-used simple diluent such as water or liquid paraffin. Preparations for parenteral administration include sterilized aqueous solutions, non-aqueous solvents, suspensions, emulsions, lyophilized formulations, and suppositories. As a non-aqueous solvent or suspension, propylene glycol, polyethylene glycol, a vegetable oil such as olive oil, or an injectable ester such as ethyl oleate may be used.

The pharmaceutical composition according to the present invention is administered at a pharmaceutically effective amount. The “pharmaceutically effective amount” used herein refers to an amount sufficient for treating a disease at a reasonable benefit/risk ratio applicable to medical treatment, and an effective dosage may be determined by parameters including the type and severity of a patient's disease, drug activity, sensitivity to a drug, administration time, an administration route and an excretion rate, the duration of treatment and drugs simultaneously used, and other parameters well known in the medical field.

The composition according to the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without side effects, and such a dose may be easily determined by one of ordinary skill in the art.

The pharmaceutical composition of the present invention may be administered into a subject via various routes. All administration routes may be considered, and the pharmaceutical composition of the present invention may be administered by, for example, oral administration, subcutaneous injection, intraperitoneal administration, intravenous, intramuscular or intrathecal injection, sublingual administration, buccal administration, rectal insertion, vaginal insertion, ocular administration, ear administration, nasal administration, inhalation, spraying through the mouth or nose, skin administration, or transdermal administration.

The pharmaceutical composition of the present invention is determined according to the type of drug as an active ingredient as well as various related parameters such as a disease to be treated, an administration route, a patient's age, sex, body weight, and the severity of a disease.

In addition, the present invention provides a method of preparing a biomimetic organ model capable of simulating the vasculopathy in Fabry disease, the method including: coating a culture container with decellularization kidney extracellular matrix: placing stem cells in which the GLA gene is knocked out in the coated culture container: adding Matrigel; and differentiating the stem cells into kidney organoids using a composition for organoid differentiation.

The “Fabry disease” used herein is one of lysosomal storage disorders that are caused by the lack or absence of GLA. Fabry disease is inherited from an X-linked recessive trait and commonly exhibits in men, while in women, relatively mild symptoms are exhibited. The lack or absence of GLA leads to lysosomal globotriaosylceramide (Gb3) accumulation, causing serious complications such as peripheral neuropathy, skin, kidney disease, cardiomyopathy, and stroke.

In the present invention, the knock-out may be performed using the CRISPR-Cas9 gene editing system, but the present invention is not limited thereto.

The “CRISPR-Cas9” or “CRISPR-Cas9 gene editing system” used herein refers to a genome editing method called a Clustered regularly interspaced short palindromic repeat (CRISPR) genetic scissors, consisting of RNA specifically binding to a specific base sequence (gRNA) and the Cas9 protein, which serves as scissors cutting a specific base sequence. When such a CRISPR/Cas9 system is used, knock-out, which suppresses the function of a specific gene by introducing plasmid DNA into cells or an animal, is possible.

The “knock-out” used herein refers to partial, substantial, and complete deletion, silencing, inactivation, or down-regulation of a gene.

The “biomimetic organ model” used herein refers to one that simulates the physiological environment where actual human organs operate, and in the present invention, may be a kidney organoid, but the present invention is not limited thereto.

The terms used in the present invention have been selected from general terms that are currently and widely used as much as possible while considering the functions of the present invention, but these may vary depending on the intentions of those skilled in the art, precedents, and the emergence of new technologies. In addition, in certain cases, there are also terms arbitrarily selected by the applicants, and in this case, their meanings will be described in detail in the relevant description of the invention. Therefore, the terms used in the present invention should be defined based on the meanings of the terms and the content throughout the present invention rather than simply the names of terms utilized in the present invention.

Throughout the specification, when one part “includes” a component, it means that it may also include other components rather than excluding components unless specifically stated otherwise. The term “approximately” or “substantially” used herein is utilized at, or in proximity to, indicated numerical values when allowable manufacturing and material tolerances, which are inherent in the meanings, are presented. These terms are used to prevent the unfair use of the disclosures in which correct or absolute values are cited to help in understanding the present invention by unscrupulous infringers.

Throughout the specification, the term “combination thereof” included in the Markush-type expression refers to a mixture or combination of one or more selected from the group consisting of constituents described in the Markush-type expression, that is, one or more selected from the group consisting of the components.

### MODE FOR INVENTION

Hereinafter, preferred examples are presented to allow the present invention to be better understood. However, the following examples are merely provided to more easily understand the present invention, and the content of the present invention is not limited by the following examples.

### Experimental Examples

### Experimental Example 1. Kidney decellularization

Pig kidney tissue was sliced to a thickness of 0.1 to 0.3 mm and washed three times for 30 minutes with distilled water. Subsequently, the slices were treated with 0.5% Triton X-100 (Sigma-Aldrich) dissolved in 1M NaCl (Samchun Pure Chemicals) for 16 hours. The slices were then washed three times more for 1 hour. To remove remaining cell components, DNase was added at 37° C. for 6 to 7 hours. After the DNase-treated tissue sections were washed with PBS for 12 hours, they were sterilized with a 0.1% peracetic acid solution for 1 hour, and then washed again with distilled water. The decellularized tissue was lyophilized at −80° C., and then used in biochemical characterization and the preparation of a kidney dECM hydrogel.

### Experimental Example 2. Preparation of Kidney dECM Hydrogel

The preparation of a kidney dECM hydrogel was performed by dissolving the previously decellularized kidney tissue with an acetic acid solution. The decellularized kidney tissue and pepsin were added to the acetic acid solution at a weight ratio of 10:1 and stirred for 72 to 96 hours depending on the concentration of decellularized tissue in the solution. After the completion of dissolution, it was neutralized using sodium hydroxide, and diluted with distilled water to prepare a kidney dECM hydrogel at a finally required concentration.

### Experimental Example 3. Kidney Organoid Differentiation

The CMC11 iPSC cell line was provided from the Catholic University of Korea (male donor). Cells at passage number between 30 and 60 were used, and the differentiation into kidney dECM-free kidney organoids was conducted as previously described. In brief, hPSCs were plated on a 24-well glass plate (LabTek) coated with 3% GelTrex (Thermo Fisher Scientific) in an mTeSRI medium (Stem Cell Technologies)+10 μM Y27632 (LC Laboratories) at a density of 5,000 cells/well (day-3). The medium was replaced with 1.5% GelTrex-containing mTeSRI on day-2, mTeSRI on day-1, and RPMI (Thermo Fisher Scientific)+11 μM CHIR99021 (Tocris) on day 0, and RPMI+B27 supplement (Thermo Fisher Scientific) on day 1.5. Afterward, to promote kidney organoid differentiation, B27 supplement-containing RPMI (Thermo Fisher Scientific) medium was supplied every 2 to 3 days.

For kidney organoid differentiation based on kidney dECM, two types of protocols were developed. The two types of protocols generally followed Freedman's protocol (Nature Communications 2015, 6 (1), 1.), but were modified as follows.

In Protocol A, after each well of a 24-well plate was coated with diluted kidney dECM (0.1%), human iPSCs, which were dissociated and undifferentiated, were uniformly placed therein. After 24 hours, human iPSCs were sandwiched between the lower layer of kidney dECM and the upper layer of Matrigel by adding 1.5% Matrigel. On day 3, to promote the kidney organoid differentiation, CHIR was treated. Afterward, an RPMI (Thermo Fisher Scientific) medium containing a B27 supplement was supplied every 2 to 3 days.

While Protocol B was largely similar to Protocol A, to enhance a vascular network, VEGF (10 ng/ml, 20 ng/ml, 30 ng/ml, and 50 ng/ml each) was treated on days 6, 9, 12, and 14, and to enhance podocyte differentiation, 10 μM Compound 1 was treated on day 12.

### Experimental Example 4. Immunofluorescent Analyses

For immunofluorescent analysis, organoids were fixed on day 18 unless otherwise noted. Specifically, for fixation, equal volumes of PBS (Thermo Fisher Scientific) and 8% paraformaldehyde (Electron Microscopy Sciences) were added to a medium for 15 minutes, and then the sample was washed with PBS three times. Subsequently, the fixed organoid culture was blocked with 5% donkey serum (Millipore)+0.3% Triton-X-100/PBS. Afterward, the blocked organoid culture was incubated in PBS containing 3% bovine serum albumin (Sigma)+ primary antibodies [anti-acetylated tubulin (Sigma-Aldrich T7451, 1:200), anti-collagen IV (Southern Biotech 1340-01, 1:300), anti-cytokeratin 8 (Abcam ab9023, 1:200), anti-GATA3 (R&D AF2605, 1:200), anti-Gb3 (TCI A2506, 1:200), anti-GLA (Thermo Fisher Scientific PA5-27349, 1:1000), anti-laminin (Sigma-Aldrich L9393. 1:200), anti-LTL (Vector Labs FL-1321, 1:200 dilution), antiMECA32 (BD Pharmingen 555849, 1:200), anti-NPHS1 (R&D AF4269, 1:200), anti-Pecaml (Abcam ab9498, 1:200), anti-TUBA4A (Abcam ab24610, 1:200), and anti-WT1 (Abcam ab89901, 1:200)] overnight, washed, and then incubated with Alexa Fluor secondary antibodies (Invitrogen). Afterward, the resulting organoids were washed and stained with DAPI, or mounted using Vectashield H-1000. Images were obtained using a Zeiss LSM 700 confocal microscope (Carl Zeiss, Germany) and ZEN 3.1 software.

### Experimental Example 5. Transmission Electron Microscope (TEM) Analysis

An adult mouse kidney block sample, a transplanted kidney organoid sample, and an in vitro kidney organoid sample were fixed with 4% paraformaldehyde and 2.5% glutaraldehyde in a 0.1M phosphate-buffered solution overnight at 4° C. After being washed with 0.1 M phosphate buffer, each sample was post-fixed with 1% osmium tetroxide in the same buffer at 4° C. for 1 hour. Subsequently, the samples were dehydrated with a series of graded ethyl alcohol solutions, exchanged with acetone, and then embedded in Epon 812. Afterward, ultrathin sections (70-80 nm) were obtained using an ultramicrotome (Ultramicrotome, Leica Ultracut UCT, Germany). The ultrathin sections were stained with both uranyl acetate and lead citrate, and observed with a transmission electron microscope (JEM 1010, Japan) at 60 kV. For quantitative analysis, 20 low-magnification (×6,000) fields were randomly selected from each thin section of the cortex, and the number of autophagosomes per 100 μm² was determined.

For a correlative light and electron microscopic study, vibratome thin sections were cryoprotected with 2.3 M sucrose at 0.1 MPB and frozen in liquid nitrogen. Semi-thin cryosections (2 μm thick) were cut at −100° C. using a glass cutter on an FC7 cryochamber (Leica)-equipped Leica EM UC7 ultramicrotome. The thin sections were labeled overnight at 4° C. using mouse polyclonal antibodies against PCAM1 and WT1. Antibody staining was visualized using Alexa Fluor secondary antibodies (Invitrogen). In addition, the thin sections were counterstained with DAPI for 10 minutes. Coverslip thin sections were observed under a confocal microscope, photographed at ×200 or ×400 magnification using a differential interference contrast setting to identify specific areas to be observed later using an electron microscope. After lifting the coverslip from the section, silver enhancement was performed for 3 minutes using an HQ silver enhancement kit (Nanoprobes), and tissue for EM was prepared as described above.

### Experimental Example 6. Real-Time Quantitative PCR Analysis

Kidney organoid samples were collected, and total RNA was isolated from each sample using a RNAiso Plus kit (Takara, Japan) according to the manufacturer's instructions. Complementary DNA was synthesized using a Maxima First Strand cDNA synthesis kit for RT-qPCR (Thermo Fisher Scientific (USA). Gene expression was analyzed with Power SYBR Green PCR Master Mix (Applied Biosystems, USA) using real time PCR (Applied Biosystems, Foster City, CA). Primer sequences used in the experiment are shown in Table 1 below. All qRT-PCRs were performed three times, and a relative mRNA expression level was determined using a 2^{-ΔΔCt} method.

TABLE 1

| | | SEQ. ID. |
|---|---|---|
| | Forward/Reverse | NO: |
| ABCB1 | F-5′ CCATGCTCAGACAGGATGTG | 1 |
| | R-5′ TTCCTGTCCCAAGATTTGCT | 2 |
| ATP1A1 | F-5′ CCAATTGTGTTGAAGGCACC | 3 |
| | R-5′ CCGTGATGATGTGGATAAAATGT | 4 |
| LRP2 | F-5′ TGTGATGCAGCCATCGAACT | 5 |
| | R-5′ TGCATTTGGGGAGGTCAGTC | 6 |
| MCAM | F-5′ CGTCTCGTAAGAGCGAACTTG | 7 |
| | R-5′ CGATGTATTTCTCTCCCTGGTC | 8 |
| PDGFR-β | F-5′ TGCAGACATCGAGTCCTCCAAC | 9 |
| | R-5′ GCTTAGCACTGGAGACTCGTTG | 10 |
| PECAM1 | F-5′ TCATTACGGTCACAATGACGA | 11 |
| | R-5′ GAGTATCTGCTTTCCACGGC | 12 |
| PKD1 | F-5′ AACAAGTCTTTGGCCATCAC | 13 |
| | R-5′ TACTCGTTCAGCACGGTGAC | 14 |
| PKD2 | F-5′ TCTTGCCAATTTCAGCCTTT | 15 |
| | R-5′ GCACAACGATCACAACATCC | 16 |
| PKHD1 | F-5′ CCATTCTCTGCCAGGTTAGC | 17 |
| | R-5′ ACCCCTAATCAGCACAGTGG | 18 |
| SLC34A1 | F-5′ TCACGAAGCTCATCATCCAG | 19 |
| | R-5′ TTCCTCAGGGACTCATCACC | 20 |

### Experimental Example 7. Statistical Analysis

For all quantitative measurements, the entire population was used to calculate statistical significance, and the mean value with an n value of 3 was used to calculate standard errors and graphical confidence intervals. Data was then analyzed using the Mann-Whitney test or Kruskal-Wallis test to determine significance between groups. In each graph, error bars represent −2 standard error of the mean (SEM), which is a confidence interval of 95. A single asterisk is used for p<0.05, two asterisks are used for p<0.01, and three asterisks are used for p<0.001.

### Examples

### Example 1. Gelation and Biochemical Characterization of Kidney dECM Hydrogel

To optimize the process of producing a kidney dECM hydrogel, a 5-step protocol was designed: (i) obtaining pig kidney tissue: (ii) decellularization: (iii) lyophilization: (iv) digestion; and (v) neutralization. As a result, cellular components were successfully removed.

### Example 2. Protocols for Differentiation of Human Kidney Organoids Based on Kidney dECM Hydrogel

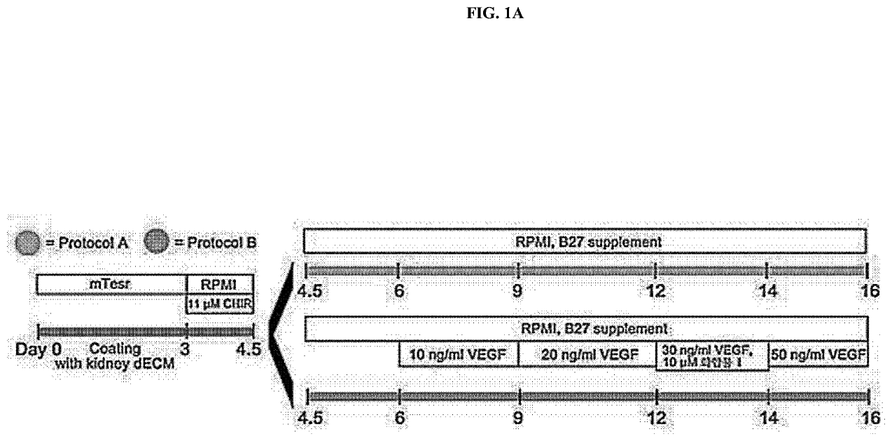

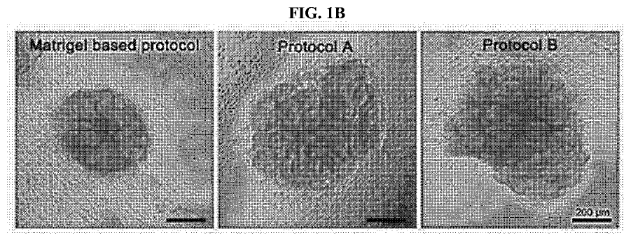

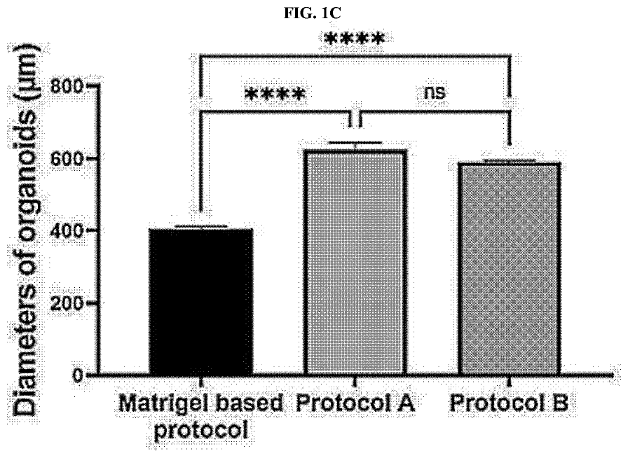

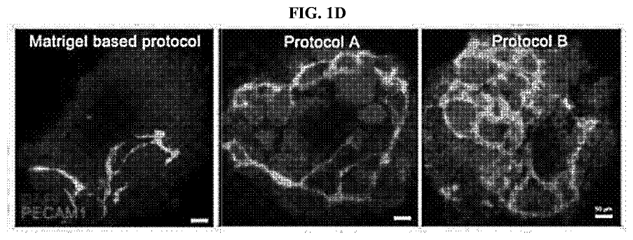

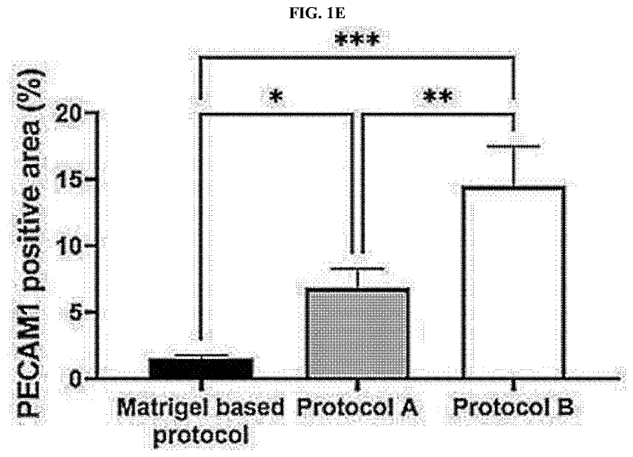

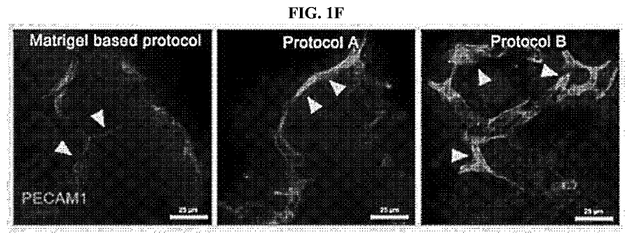

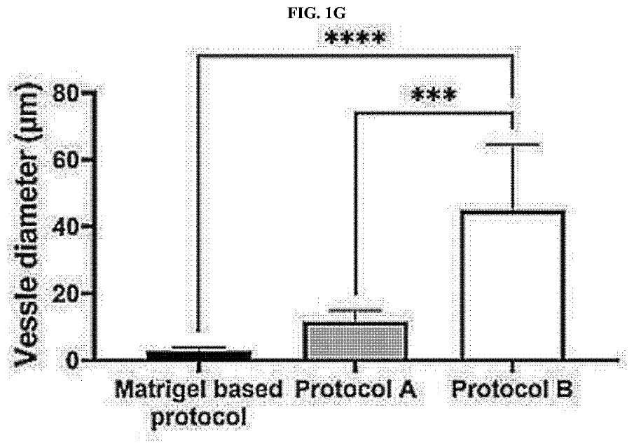

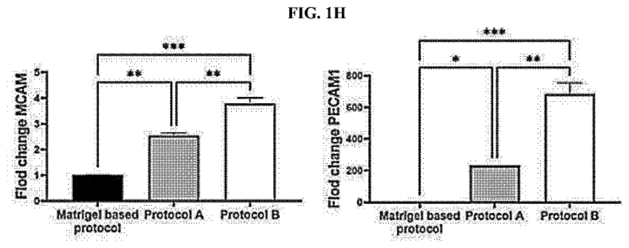

To confirm the effect of kidney dECM on kidney organoid differentiation, two 3D culture systems for differentiating human iPSCs into kidney organoids using kidney dECM hydrogels were developed. Each culture system is shown in FIG. 1A.

As shown in FIG. 1A, in Protocol A, each well of a 24-well plate was coated with 0.1% (w/v) kidney dECM diluted in phosphate-buffered saline (PBS), and human iPSCs, which are dissociated and undistributed, were uniformly placed therein. After 24 hours, the hPSCs were sandwiched between the lower layer of kidney dECM and the upper layer of Matrigel by adding 1.5% Matrigel. The sandwiched hPSCs were formed into a dense ball-shaped colonies within 48 hours, and then internal cavities developed. Afterward, on day 3, the cells were treated with CHIR, and as shown in FIG. 1, to promote kidney organoid differentiation, an appropriate medium was supplied every 2 to 3 days.

In addition, as shown in FIG. 1A, while Protocol B was initiated in the same manner as Protocol A, to enhance a vascular network, a vascular endothelial growth factor (VEGF) was added on days 6, 9, 12, and 14, and to enhance the differentiation of podocytes, which are cells constituting the visceral layer of the glomerular capsule of the kidney, on day 12, Compound 1, which is a TGF-β inhibitor, was added.

### Example 3. Confirmation of Enhancing Effect of Kidney dECM Hydrogel on Formation of Vascular Network in Human Kidney Organoids

To determine the effect of kidney dECM hydrogel on kidney organoid differentiation, kidney organoids were produced using Protocols A and B, and compared to the phenotypes of kidney organoids (control kidney organoids) produced using kidney dECM-free Matrigel. The results are shown in FIGS. 1B and 1C.

As shown in FIGS. 1B and 1C, bright field microscopy showed that the kidney organoids produced by Protocols A and B have a tubular structure, and the organoid size thereof is larger than that of the control kidney organoid.

The effect of a kidney dECM hydrogel on the formation of a vascular network that broadly surrounds a nephron-like structure in kidney organoids was confirmed through confocal images. The results are shown in FIGS. 1D to 1G.

As shown in FIGS. 1D to 1G, the formation of a platelet endothelial cell adhesion molecule 1 (PECAM1)⁺ vascular network was limited in the control organoids, was increased in the kidney organoids produced by Protocol A, and further increased in those produced by Protocol B. In addition, the area and diameter of PECAM1⁺ vasculature were improved by Protocol A, and further improved by Protocol B.

In addition, through RT-qPCR, the gene expression of the vascular marker PECAM1 and its precursor MCAM was confirmed. The results are shown in FIG. 1H.

As shown in FIG. 1H, according to Protocols A and B, compared to the gene expression of the control kidney organoids, it was confirmed that gene expression of the vascular marker PECAM1 and its precursor MCAM was upregulated.

### Example 4. Confirmation of Effect of Kidney dECM Hydrogel on Improving Glomerular Angiogenesis and Kidney Organoid Morphogenesis

4.1. Confirmation of Effect of Kidney dECM Hydrogel on Glomerular Angiogenesis

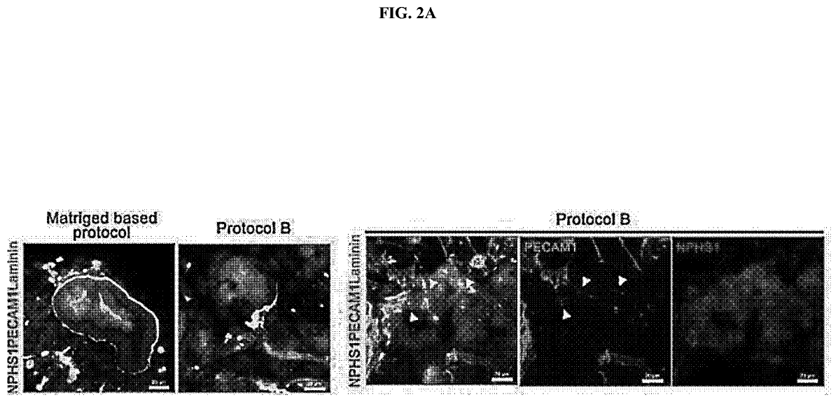

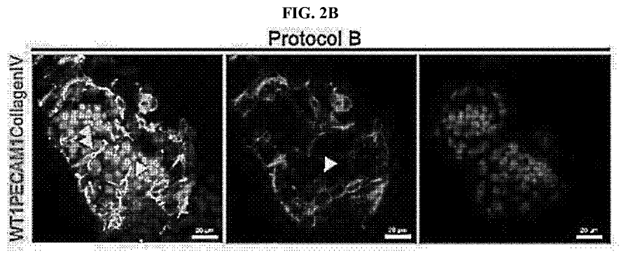

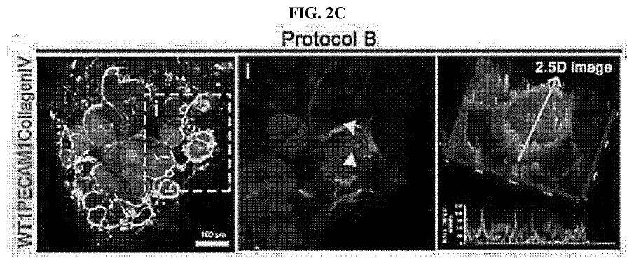

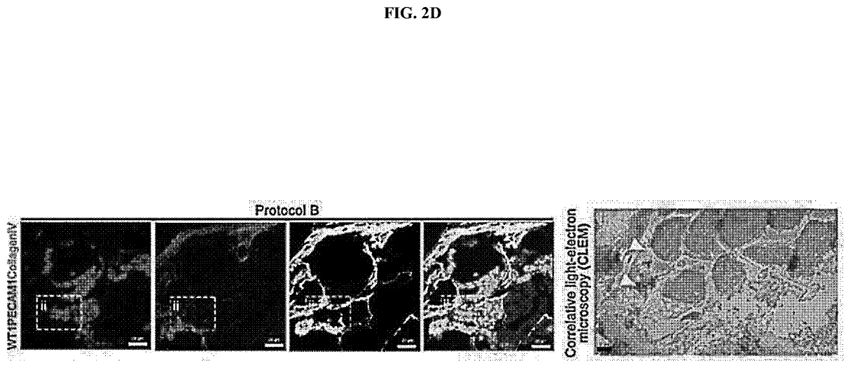

It was confirmed whether the kidney dECM-induced vascularization of organoids in vitro extended to the glomerular compartment. Specifically, in the kidney organoids cultured using Protocols A and B, NPHS1⁺ or WT1⁺ podocyte clusters invaded by PECAM1⁺ vasculature were confirmed through confocal images. The results are shown in FIGS. 2A to 2C.

As shown in FIG. 2A, even when Protocol A was used, compared to the control, vascularization surrounding the glomerular structure increased, but PECAM1⁺ vascular invasion into the glomerular structure was hardly observed. Meanwhile, when Protocol B in which VEGF and Compound 1 were added to Protocol A was used, it was confirmed that vascularization surrounding the glomerular structure was significantly improved, a PECAM1⁺ vessel-like structure with a larger diameter was confirmed.

As shown in FIGS. 2A to 2C, it was confirmed that a small branch of the larger PECAM1⁺ vessel-like structure invades the NPHS1⁺ or WT1⁺ glomerular structure.

In addition, to confirm the PECAM1+ vasculature invasion into the glomerular structure, a correlative light- and electron-microscopy (CLEM) study was conducted by overlaying an PECAM1-, WT1-, and collagen IV-stained confocal microscope image with an electron microscope (EM) image of the same structures in the kidney organoids. The results are shown in FIG. 2D.

As shown in FIG. 2D, through the overlay of the confocal microscope and EM images, it was confirmed that PECAM1+ endothelial cells infiltrated into WT1+ podocyte clusters.

4.2. Confirmation of Enhancing Effect of Kidney dECM Hydrogel Kidney Organoid Morphogenesis

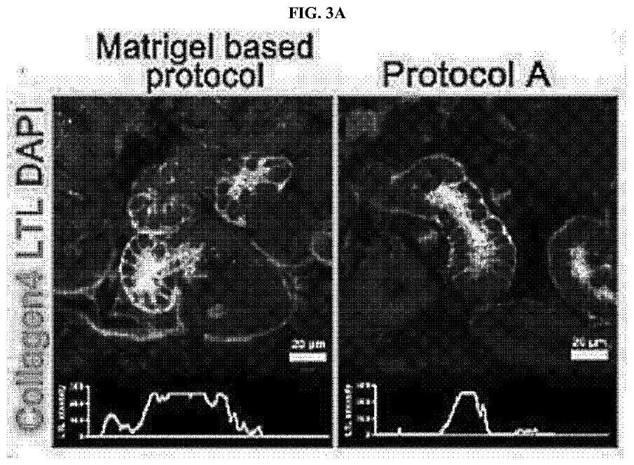

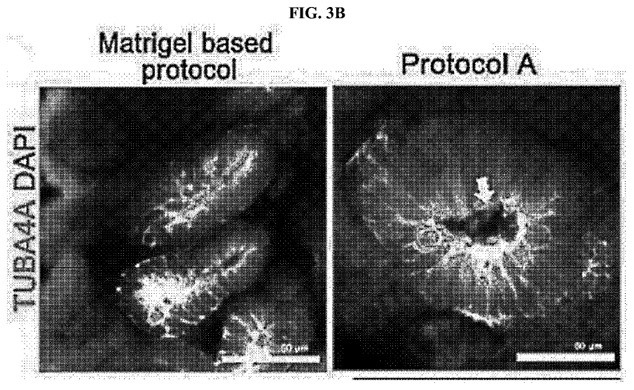

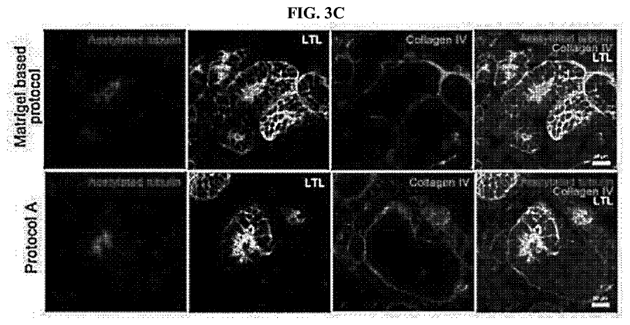

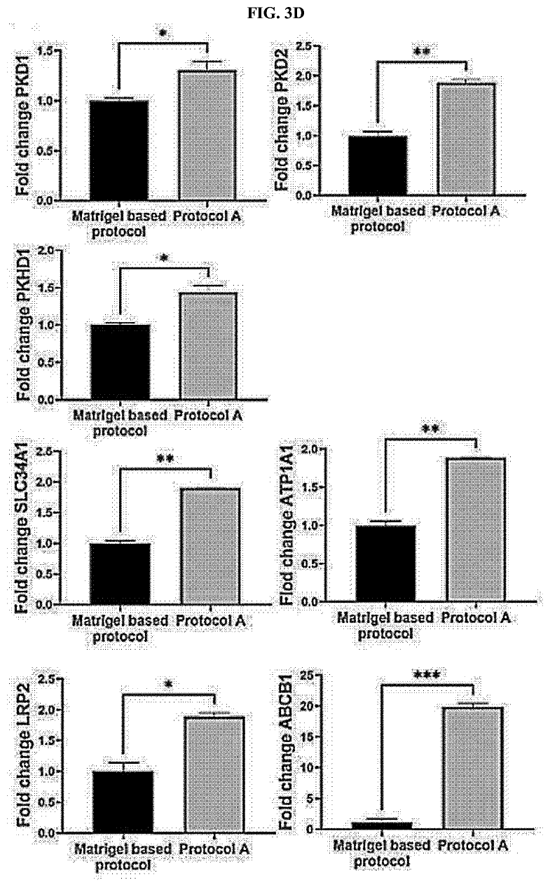

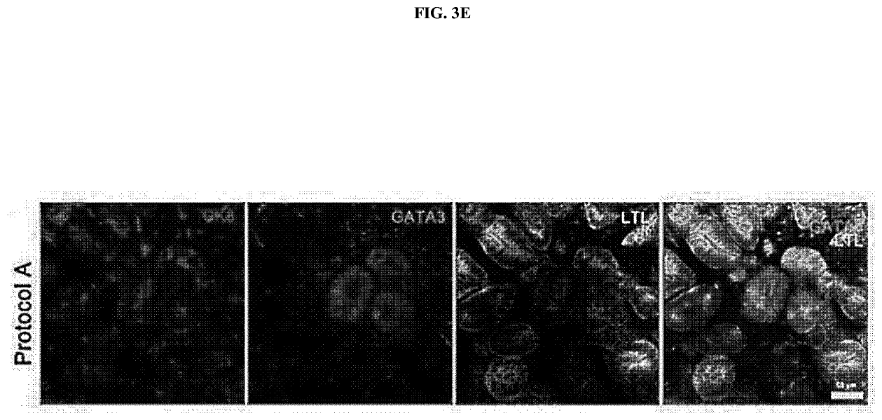

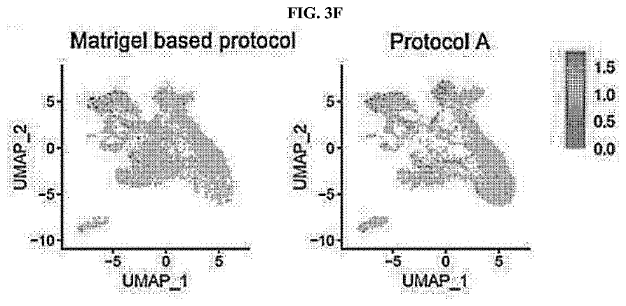

When kidney organoids are transplanted into a mouse kidney, cultured in a microfluidic system, or transplanted into a chick chorioallantoic membrane, the progressive morphogenesis of tubular structures can be confirmed along with increased vascularization of kidney organoids. Accordingly, the present inventors speculated that, when vascularization is enhanced by a dECM-based culture system, progressive morphogenesis and maturation of kidney organoids would occur in human iPSC-derived tubular cells of kidney organoids in vitro. Accordingly, confocal images were confirmed through immunofluorescent staining of LTL, collagen IV, TUBA4A, and acetylated tubulin in kidney organoids. The results are shown in FIGS. 3A to 3C.

As shown in FIGS. 3A to 3C, the enhanced polarization of proximal tubules, along with apical enrichment of the brush border marker, lotus tetragonolobus lectin (LTL), and primary cilia, was confirmed in kidney organoids cultured with kidney dECM.

In addition, the expression of ciliary genes (PKD1, PKD2, and PKHD1), tubular epithelial transport genes (SLC34A1 and ATP1A1), and drug transport genes (ABCB1 and LRP2) was confirmed through qRT-PCR. The results are shown in FIG. 3D.

As shown in FIG. 3D, consistent with polarization, the expression of ciliary genes (PKD1, PKD2, and PKHD1), tubular epithelial transport genes (SLC34A1 and ATPIA1), and drug transport genes (ABCB1 and LRP2) were upregulated in the kidney organoids cultured with kidney dECM. This means that the functional potential of the kidney organoids was improved.

ECM and ECM-related proteins play an important role in branching morphogenesis and maturation of the UB in kidney development by providing a physical substrate for the spatial organization of cells, releasing growth factors, and regulating a signaling pathway such as an integrin pathway. In addition, the kidney dECM of the present invention contains a glial cell-derived neurotrophic factor (GDNF), which is an important cytokine in branching morphogenesis of UB cells. Accordingly, the inventors confirmed whether kidney dECM contributed to UB induction in kidney organoids.

In addition, confocal images were confirmed through immunofluorescent staining of CK8, GATA3, and LTL in kidney organoids. The results are shown in FIG. 3E.

As shown in FIG. 3E, high expression of the anterior intermediate mesoderm (AIM) marker, GATA3, in kidney organoids differentiated using Protocol A was confirmed through immunofluorescent analysis.

In addition, the GATA3 expression level was confirmed through uniform manifold approximation and projection (UMAP). The results are shown in FIG. 3F.

As shown in FIG. 3F, through single-cell RNA sequencing (scRNAseq), an increase in the GATA3+ cell population in the kidney organoids differentiated using Protocol A was confirmed. This means that kidney dECM is effective in differentiation of AIM in kidney organoids, which is the early stage of UB development.

Taking the above results together, kidney dECM has excellent effects in improving glomerular vascularization and tubular compartment maturation in kidney organoids.

### Example 5. Recapitulation of Fabry Nephropathy with Vasculopathy Using Vascularized Kidney Organoids and CRISPR-Cas9 Gene Editing

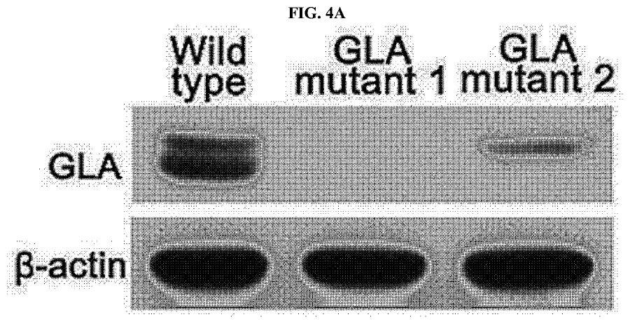

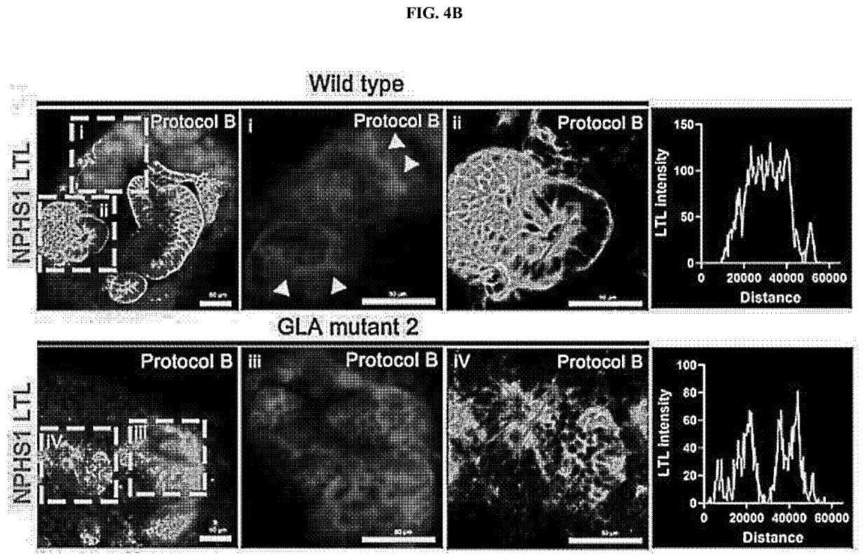

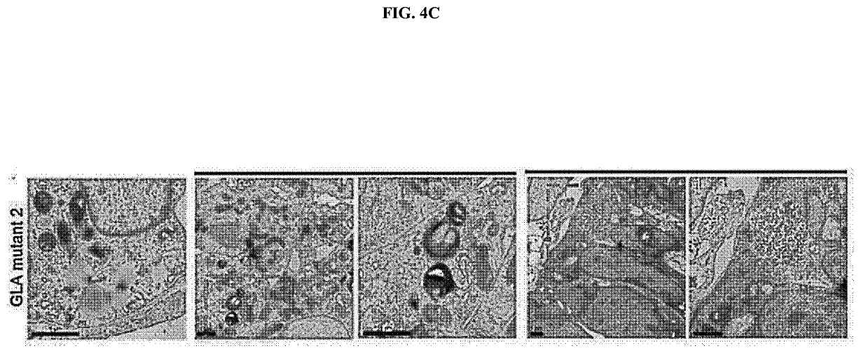

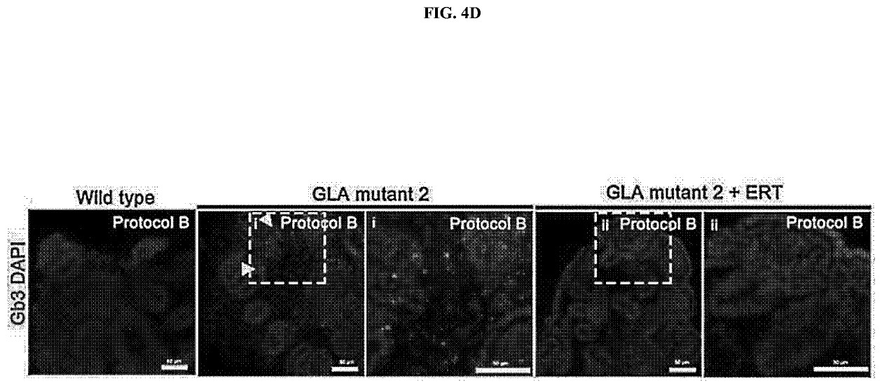

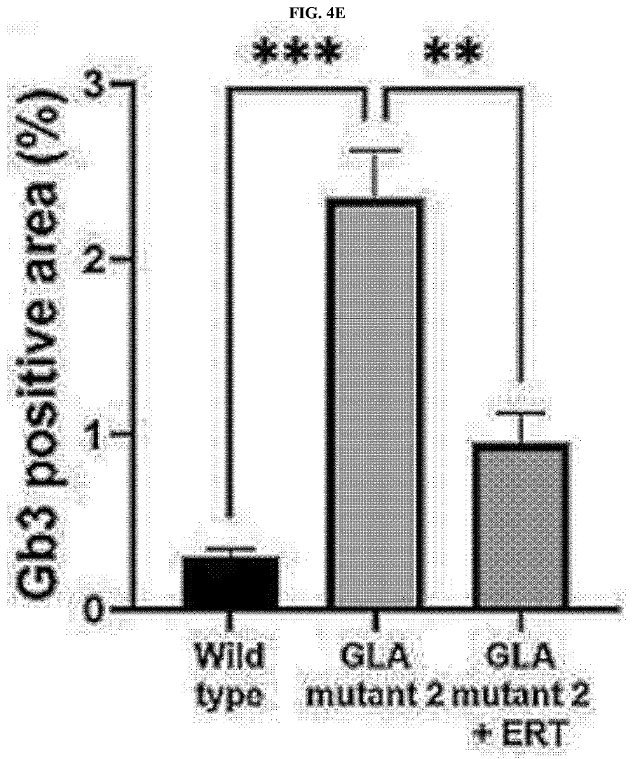

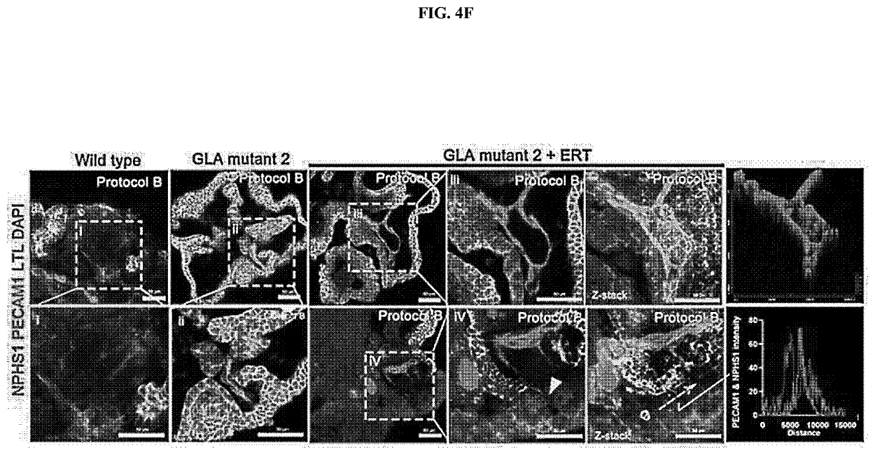

By transfection with an all-in-one vector expressing Cas9, GLA-specific single-guide RNA and GFP, two clones of GLA mutant human iPSCs (GLA mutant 1 and GLA mutant 2) were made. Then, the GLA mutant human iPSCs were differentiated into kidney organoids using Protocol B. Afterward, GLA expression in GLA mutant 1 and GLA mutant 2 was confirmed through Western blotting. The results are shown in FIG. 4A.

As shown in FIG. 4A, GLA protein could not be confirmed in kidney organoids differentiated from the GLA mutant 1 human iPSCs, and GLA protein levels were reduced in kidney organoids differentiated from the GLA mutant 2 human iPSCs. This is because the structural abnormalities in kidney organoids differentiated from the GLA mutant 1 human iPSCs were too severe for phenotypic analysis, whereas the structural abnormalities in kidney organoids differentiated from the GLA mutant 2 human iPSCs were relatively minor and could be subjected to analysis. Therefore, the kidney organoids differentiated from the GLA-mutant 2 human iPSCs were used as a Fabry disease model.

The wild-type and GLA mutant kidney organoids were subjected to immunofluorescent staining analyses of NPHS1 and LTL. The results are shown in FIG. 4B.

As shown in FIG. 4B, the linear pattern of tracks of NPHS1 expressed on the basolateral side of podocytes was disorganized in the GLA mutant kidney organoids. In addition, the disruption of tubular polarity was confirmed in the GLA mutant kidney organoids.

The accumulation of lipid droplets, glycoproteins, and zebra bodies was confirmed through TEM images. The results are shown in FIG. 4C.

As shown in FIG. 4C, the formation of abundant lipid droplets, electron-dense granular deposits, and electron-dense lamellate lipid-like deposits form concentric bodies (zebra bodies) in the cytoplasm of podocytes and tubules was observed in GLA mutant kidney organoids, which is compatible with the phenotype of Fabry disease in human kidneys.

Immunofluorescent staining analysis of Gb3 was performed on a wild type, GLA mutant kidney organoids, and human recombinant agalsidase-α-treated GLA mutant kidney organoids, and a Gb3-positive area was confirmed graphically. The results are shown in FIGS. 4D and 4E.

As shown in FIGS. 4D and 4E, Gb3 accumulation was prominent in the GLA mutant kidney organoids, and this decreased after being treated with agalsidase-α by recombinant enzyme replacement therapy (ERT). This shows that the GLA mutant kidney organoids efficiently recapitulated Fabry disease.

Next, the phenotype of the vasculopathy in Fabry disease was determined using GLA mutant kidney organoids produced by Protocol B. Immunofluorescent staining analyses of NPHS1, PECAM1, and LTL were conducted on the wild type, GLA mutant kidney organoids, and human recombinant agalsidase-α-treated GLA mutant kidney organoids. The results are shown in FIG. 4F.

As shown in FIG. 4F, vascularization was limited in the GLA-mutant human iPSC kidney organoids, whereas the wild-type kidney organoids were highly vascularized. Meanwhile, ERT using recombinant human agalsidase-α restored the vascular network through glomerular angiogenesis and the formation of large lumen vessels in GLA mutant kidney organoids.

The above results demonstrate that the method of producing vascularized kidney organoids in combination with the CRISPR-Cas9 gene editing system is useful in modeling a disease such as the vasculopathy in Fabry disease and development of treatment options.

It should be understood by those of ordinary skill in the art that the above description of the present invention is exemplary, and the exemplary embodiments disclosed herein can be easily modified into other specific forms without changing the technical spirit or essential features of the present invention. Therefore, it should be interpreted that the exemplary embodiments described above are illustrative in all aspects, and not restrictive.

### INDUSTRIAL APPLICABILITY

The present inventors invented two kinds of protocols for preparing kidney organoids by placing stem cells between kidney decellularized extracellular matrix and Matrigel and uniquely treating a composition for kidney organoid differentiation, and since it was confirmed that these protocols not only promote the formation of a vascular network and glomerular angiogenesis in kidney organoids, but also exhibit the excellent effect of maturing kidney organoids, it is expected that the kidney organoids prepared according to the present invention can be effectively used in disease modeling, drug screening, and regenerative medicine.

1. A method of preparing highly differentiated kidney organoids, comprising:
coating a culture container with kidney decellularized extracellular matrix (dECM);
placing stem cells in the coated culture container;
adding Matrigel; and
differentiating the stem cells into kidney organoids using a composition for organoid differentiation.

2. The method of claim 1, wherein the differentiating of the stem cells into kidney organoids includes adding a glycogen synthase kinase 3 (GSK3) inhibitor and providing a medium for differentiation.

3. The method of claim 1, wherein the differentiating of the stem cells into kidney organoids includes adding a vascular endothelial growth factor (VEGF); and adding a TGF-β inhibitor.

4. The method of claim 1, wherein the stem cells are placed between the kidney dECM and the Matrigel.

5. The method of claim 1, wherein the coating is to coat each well of the well plate with kidney dECM.

6. The method of claim 1, wherein the method satisfies the following characteristics:
(a) promotion of the formation of a vascular network of kidney organoids;
(b) promotion of glomerular angiogenesis;
(c) promotion of kidney organoid morphogenesis; and
(d) enhancement of kidney organoid maturity.

7. The method of claim 1, wherein the method increases the expression of one or more genes selected from the group consisting of platelet endothelial cell adhesion molecule 1 (PECAM1), a melanoma cell adhesion molecule (MCAM), polycystin 1 (PKD1), polycystin 2 (PKD2), polycystic kidney and hepatic disease 1 (PKHD1), solute carrier family 34 member 1 (SLC34A1), ATPase Na+/K+ transporting subunit alpha 1 (ATP1A1), ATP binding cassette subfamily B member 1 (ABCB1), LDL receptor related protein 2 (LRP2), and GATA binding protein 3 (GATA3).

8. The method of claim 1, wherein the stem cells are pluripotent stem cells.

9. The method of claim 1, wherein a kit is used in the method, said kid is for preparing highly differentiated kidney organoids, comprising a first kit, a second kit, and instructions,
wherein the first kit includes kidney decellularized extracellular matrix (dECM), stem cells, and Matrigel, the second kit includes a composition for organoid differentiation, and the instructions include a step of sequentially treating the first and second kits.

10. The method of claim 9, wherein the composition for organoid differentiation includes two or more selected from the group consisting of a glycogen synthase kinase 3 (GSK3) inhibitor, a vascular endothelial growth factor (VEGF), a TGF-β inhibitor, and a medium for differentiation.

11. Highly differentiated kidney organoids, which are prepared by the method of claim 1 and satisfy the following characteristics.
(a) promotion of the formation of a vascular network of kidney organoids;
(b) promotion of glomerular angiogenesis;
(c) promotion of kidney organoid morphogenesis; and
(d) enhancement of kidney organoid maturity.

12. A method of preparing a biomimetic organ model capable of simulating the vasculopathy in Fabry disease, comprising:
coating a culture container with decellularization kidney extracellular matrix;
placing stem cells in which the alpha-galactosidase (GLA) gene is knocked out in the coated culture container;
adding Matrigel; and
differentiating the stem cells into kidney organoids using a composition for organoid differentiation.
